(19) United States
(12) Patent Application Publication
(10) Pub. No.: US 2026/0151514 A1
(43) Pub. Date: Jun. 4, 2026

(54) PHOTOACOUSTIC VAPORIZATION OF EXOSKELETAL AND ENDOSKELETAL DROPLETS

(71) Applicant: The Regents of the University of Colorado, a body corporate, Denver (CO)

(72) Inventors: Mark Borden, Boulder, CO (US); Anish Silwal, Boulder, CO (US); Gazendra Shakya, Boulder, CO (US)

(21) Appl. No.: 19/126,513

(22) PCT Filed: Nov. 3, 2023
(86) PCT No.: PCT/US2023/078694

§ 371 Date: May 1, 2025

Related U.S. Application Data
(60) Provisional application No. 63/422,388, filed on Nov. 3, 2022.

(51) Int. Cl. A61K 49/22; A61K 41/00; A61K 47/06

(52) U.S. Cl. CPC A61K 49/225; A61K 41/0052; A61K 47/06

(57) ABSTRACT

The inventive technology is directed to novel photoacoustic endoskeletal droplet compositions and methods of making the same. Specifically, the naphthalocyanine dye-loaded endoskeletal droplets having a solid hydrocarbon core surrounded by a fluorocarbon liquid, which can further be applied to photoacoustic imaging and sensing, as well as photothermal therapy.

### CROSS-REFERENCE TO RELATED APPLICATIONS

This International PCT application claims the benefit of U.S. Provisional Application Ser. No. 63/422,388, filed Nov. 3, 2022, the specification, claims and drawings of which are incorporated herein by reference in their entirety.

### GOVERNMENT INTEREST

This invention was made with Government support under grant number R01CA239465 awarded by the National Institutes of Health (NIH). The U.S. Government has certain rights in this invention.

### TECHNICAL FIELD

The inventive technology is directed to novel photoacoustic endoskeletal and exoskeletal droplets which can further be applied to photoacoustic imaging and sensing, as well as photothermal therapies.

### BACKGROUND

Phase-change droplets are liquid colloidal particles suspended in aqueous media that can be converted to gas-containing microbubbles. This phase transformation can be triggered in situ by various mechanisms, including thermal heating, acoustic insonation and optical irradiation. Phase-change droplets are gaining interest for energy storage, localized chemical reactions, and biomedical sensing, imaging and therapy. For the latter, utility arises from the resulting vaporized microbubble, which is acoustically responsive to extracorporeally delivered ultrasound. Optical stimulation of the vaporization event has garnered increasing interest for photoacoustic imaging and photothermal therapy.

The energy needed for droplet vaporization depends significantly on their formulation. Low molecular weight fluorocarbons (PFCs), like perfluoropropane (C₃F₈) and perfluorobutane (C₄F₁₀), can be used to formulate easily vaporizable droplets. However, these PFCs have high vapor pressure (fugacity) and thus are somewhat unstable as a colloidal suspension, especially upon injection into the body where they are exposed to atmospheric and respiratory gases. Higher molecular weight PFCs, like perfluoropentane C₅F₁₂, increase the colloidal stability, but they also require more energy to vaporize.

A novel class of droplets, known as vaporizable endoskeletal droplets, are complex emulsions containing a solid hydrocarbon phase in their inner core along with a volatile fluorocarbon as the outer liquid. Melting of the solid hydrocarbon phase interrupts intermolecular cohesion within the liquid fluorocarbon, thereby lowering the spinodal temperature and triggering vaporization of the volatile fluorocarbon. The droplet vaporization temperature can therefore be tuned by choosing an appropriate hydrocarbon melting temperature. Shakya et al. recently showed that droplet processing history can also have a significant effect on vaporization behavior. While heating and hyperthermia can be used for many applications, there is an interest in formulating new endoskeletal droplets that vaporize without the need for direct heating, such as exposure to light or sound.

Moreover, as described herein, Applicants has demonstrated the production of vaporizable endoskeletal droplets having a containing a volatile fluorocarbon as the outer liquid inner core surrounded by a solid hydrocarbon phase outer shell. These novel endoskeletal droplets exhibit vaporization characteristics that can be useful for therapeutic as well as diagnostics applications.

### SUMMARY OF THE INVENTION

The present invention is directed to novel photoacoustic endoskeletal droplets and methods of photothermal activation involving radiation of the endoskeletal droplet having a suitable photosensitive species that facilitates absorption of light to dissipate heat, leading to melting of the hydrocarbon core and vaporization of the perfluorocarbon (See FIG. 1). The vaporization event itself may launch a detectable acoustic wave, or the resulting microbubble can be detected by pulse-echo ultrasound.

In another aspect, the present invention is directed to novel photoacoustic endoskeletal droplets having a solid hydrocarbon core surrounded by a fluorocarbon liquid. In one preferred aspect, the solid hydrocarbon core can further be loaded with a naphthalocyanine dye. In another aspect, the present invention includes novel photoacoustic exoskeletal droplets having a having a solid hydrocarbon shell surrounding a fluorocarbon liquid core. In one preferred aspect, the solid hydrocarbon shell can further be loaded with a naphthalocyanine dye.

In a preferred aspect, the naphthalocyanine dye of the invention includes a hydrophobic naphthalocyanine dye chelating a Zn+ion at its core (ZnNc). The presence of the Zn ion changes the absorbance of the molecule to the NIR (near-infrared) wavelength range (˜720 nm). NIR absorption is helpful because it can penetrate tissues to a greater depth.

### BRIEF DESCRIPTION OF THE FIGURES

FIG. 1A-C: shows exemplary design concept for photoacoustic endoskeletal droplets. (A) Structure and composition of the solid-hydrocarbon (HC) in liquid-fluorocarbon (FC) droplet with dye loaded into the hydrocarbon phase. The structures of the NIR optically absorbing naphthalocyanine dye (zinc 2,11,20,29-tetra-tert-butyl-2,3-naphthalocynanine) and the HC (eicosane, n-C₂₀H₄₂) and FC (C₅F₁₂) are shown. (B) The droplet is irradiated by a laser pulse, and the dye inside the droplet absorbs the light energy. The laser heating melts the hydrocarbon core and initiates vaporization of the fluorocarbon phase. (C) Phase conversion to a microbubble makes the particle echogenic for photoacoustic and ultrasound imaging.

FIG. 2A-C: shows microfluidic device for monodisperse endoskeletal droplet synthesis. (A) Design of the microfluidic device used to synthesize the droplets. The chip consists of three inlets, one each for the HC phase (labeled as 1), FC phase (labeled as 2) and water (labeled as 3), and an outlet for collection of the droplets (labeled as 4). The device was heated using a resistance heater (8) fed by a power supply (7) to keep the HC phase in the liquid state during droplet synthesis. (B) Top-view of the microfluidic device showing the flow channels (6), inlets 1-3, outlet 4 and the flow-focusing junction (5). (C) Zoomed-in image of the region around the flow-focusing junction showing a detailed view of the inlet and outlet geometries.

FIG. 3A-B: show synthesis of endoskeletal droplets by shaking. Smaller, polydisperse endoskeletal droplets were prepared using a shaking method. (A) Synthesis of blank droplets, and (B) droplets containing zinc-naphtalocynanine dye as core content.

FIG. 4A-B: shows exemplary schematics of the photoacoustic experimental setups. (A) Optical observation of individual monodisperse droplet vaporization. (B) Photoacoustic signal measurement of polydisperse droplets.

FIG. 5A-C: show monodisperse droplet characterization. (A) Brightfield microscope image of the dye-loaded droplets in water. Inset figure shows enlarged image of a typical endoskeletal droplet with a solid HC core. Scale bar =10 μm. (B) Size distribution of a typical droplet sample showing the mean and standard deviation of three measurements. The droplets had a mean diameter of 11.7 μm with a standard deviation of ±0.7 μm (n=3). (C) Absorption spectra of the samples. The inset in 3c (blue curve) shows the difference between absorbance of the dye-loaded droplets (red curve) and the blank droplets (grey curve). An absorption peak near 720 nm was observed in the dye-loaded droplets that corresponds to increased absorption by the dye alone dispersed in water (green curve). The aqueous phase alone exhibited minimal absorption over these wavelengths.

FIG. 6A-B: show polydisperse droplet characterization. (A) A microscope image taken in brightfield mode of polydisperse droplets suspended in water. Inset figure shows close-up view of a typical droplet with hydrocarbon core. Scale bar =10 μm. (B) Size distribution of a typical polydisperse sample showing the mean and standard deviation averaged over three readings. The mean diameter was 0.75 μm with a standard deviation of ±0.026 μm.

FIG. 7A-E: shows single droplet vaporization. (A) A single dye-loaded endoskeletal droplet was placed at the center of the 720 nm laser beam excitation spot on the focal plane of the microscope objective. (B) Vaporization was triggered as the increasing fluence reached the threshold value. (C) A bubble formed upon completion of vaporization. (D) A plot for the detection 532 nm CW laser beam intensity versus the laser shot number for a typical dye-loaded droplet (red) and control droplet (green). The sharp drop in the intensity was an indication of the bubble formation through vaporization and thus blocking the laser path. For the control droplet (green curve), the detection beam intensity continued to be on the same level for all the fluences tested (0 to 100 mJ/cm2). (E) Frequency density histogram (red bars) for the vaporization of 300 dye-loaded droplets. 11 out of 300 dye-loaded droplets did not vaporize. The histogram was fitted with a Gaussian (black curve) which gave an average threshold fluence of 64.8 mJ/cm2. The green curve represents the frequency density plot for 60 blank droplets. None of the blank droplets vaporized. Scale bars=10 μm.

FIG. 8: shows photoacoustic measurement. Plot of the mean peak-to-peak voltage signal as a function of laser fluence incident on droplet samples flowing through the tube. The red curve shows response generated from endoskeletal droplets containing zinc-naphthalocyanine, and the black curve represents signal response from blank droplets.

FIG. 9A-D: show ultrasound imaging. Ultrasound images were taken of polydisperse droplets flowing in the tube exposed to 720-nm laser at a fluence of 25 mJ/cm2 (laser beam region denoted by red dotted circle) and ultrasound insonation at 14 MHz and 0.2 MI. B-mode images of the tube containing dye-loaded droplets immediately after the laser was turned on (A), and after few minutes of laser irradiation (B). (C) Cross-sectional image of the tube containing dye-loaded droplets as the laser was activated. (D) Blank droplets.

FIG. 10: diagram demonstrating exemplary exoskeleton droplet having a fluorocarbon core and outer hydrocarbon layer and an exemplary exoskeleton droplet after heating where the fluorocarbon has vaporized forming a melted hydrocarbon lens structure in one embodiment thereof.

FIG. 11A-C: Synthesis and characterization of FC/HC exoskeletal and HC/FC endoskeletal droplets. (A) Molecular structure of surfactants, phospholipid, 1,2-dibehenoyl-sn-glycero-3-phosphocholine (DBPC) and fluorosurfactant Perfluoropolyether, Krytox 157 FSH surfactants. (B) Emulsification process steps involved in synthesis of HC/FC endoskeletal droplets and microscope image of a typical endoskeletal droplet. (C) Steps involved in synthesis of FC/HC droplets. A change in type of surfactant results in distinct morphology. The exoskeletal droplets were formed by shaking FC/HC/lipid suspension using a dental amalgamator. Microscopic image of a representative exoskeletal droplet. Scale bars, 10 μm.

FIG. 12A-B: Endoskeletal vs Exoskeletal droplets: Morphology. Brightfield microscope images of representative (A) exoskeletal droplets and (B) endoskeletal suspension. Exoskeletal droplets show hydrocarbon outer shell structure with fluorocarbon entrapped as core or partially covered with hydrocarbon shell. Endoskeletal droplets have smooth spherical structure of fluorocarbon outer layer entrapping solid hydrocarbon as core. Scale bar 10 μm.

FIG. 13A-B: Exoskeletal droplets after density gradient separation using Ficol solution. Density separation method distinctly isolates droplets based on the density gradient of hydrocarbon and fluorocarbon rich droplets. (A)) shows schematic of exoskeletal droplet suspension before and after centrifugation. Right hand side top panel show distribution plot and bottom panel show corresponding microscope image of droplets pellet (B-C), FC rich pellet settled at the bottom, (D-E) HC rich supernatant containing small sized exodroplets, and (E-F) HC rich cake obtained after density centrifugation of exoskeletal droplets. Scale bar 50 μm.

FIG. 14A-E: Vaporization study of the exoskeletal droplets. (A) Schematics of the heating stage used to study droplet vaporization. Exoskeletal droplets were collected in an incubation chamber and placed on a microcontroller heating transparent stage and observed under microscope. Bottom panel shows microscopic image sequence showing vaporization of a typical exoskeletal droplet (B), as the temperature of the transparent stage was increased the hydrocarbon (HC) shell starts melting (C) resulting in formation of a gaseous bubble (D). Finally, the melted HC forms a lens like structure around the vaporized bubble as shown in (E). Scale bars, 10 μm FIG. 15A-B: Multi-site vaporization on an exoskeletal droplet. (A) Microscopic image of a typical exoskeletal droplet exposed to heat treatment. As the temperature increases a vapor bubble forms from one of the ends of the exoskeletal droplet. Further, as the temperature rises from other end, another vapor bubble seen forming from the same exoskeletal droplet. However, soon the second bubble diffuses in the first bubble and disappear completely. Finally, the wrinkled hydrocarbon shell starts melting and forms a liquid lens type structure around the bubble as shown in the last figure. Scale bars 10 μm. (B) Schematic of the steps involved in two bubble formation process.

FIG. 16A-H: Heat treatment: effect on size and vaporization temperature. Top panel shows size distribution (A) and microscope images of Ficoll separated untreated exoskeletal droplets at temperatures (B) 22.5° C. and at (C) 30° C. while (D) shows resultant image when (B) was subtracted from (C). Bottom panel shows (E) size distribution plot and microscope images of Ficol separated and heat-treated exoskeletal droplets at (F) 22.5 and (G) 30° C. The resultant image after subtracting image (F) from image (G) is shown in (H). The resultant microscopic images (D) and (H) clearly show more instances of vaporization events (bright spots) at similar temperature for heat treated exoskeletal droplets as compared to as untreated exoskeletal droplets. Scale bar 100 μm.

FIG. 17A-B: Vaporization behavior of the droplets. (a) Typical vaporization curves for Ficoll separated heat-treated and untreated exoskeletal droplets. Panel (B) shows corresponding untreated and heat-treated Ficoll separated endoskeletal droplets vaporization behavior. The as-prepared exo/endo droplets were subjected to Ficoll density separation and resultant supernatants were collected. Further, the collected supernatant were heated to 45° C. (termed as treated sample). Vaporization behavior of heat treated and untreated samples were studied under the microscope. A gaussian cumulative fraction fit was added to each plot as a solid line. The best-fit mean value were ˜29.6±0.06 and 35.1±0.15 for heat treated and untreated exoskeletal droplets, while corresponding values for heat treated and untreated endoskeletal droplets were 38.68±0.26 and 37.37±0.24, respectively. Plots in (A) are an average of 9 vaporization studies while plots in (B) are mean of 7 individual studies.

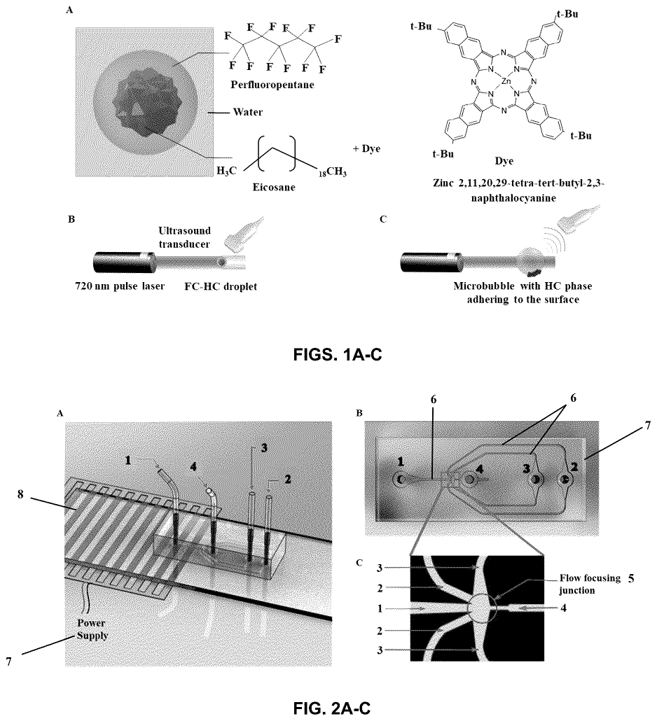

### DETAILED DESCRIPTION OF THE INVENTION

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries. The present disclosure is explained in greater detail below. This disclosure is not intended to be a detailed catalog of all the different ways in which embodiments of this disclosure can be implemented, or all the features that can be added to the instant embodiments. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the scope of the instant disclosure. Hence, the following specification is intended to illustrate some particular embodiments of the disclosure, and not to exhaustively specify all permutations, combinations, and variations thereof.

### Embodiment 1

### Vaporizable Endoskeletal Droplets

Vaporizable endoskeletal droplets are solid-hydrocarbon in liquid-fluorocarbon droplets in which melting of the hydrocarbon phase leads to vaporization of the fluorocarbon phase. Vaporization of the endoskeletal droplets has been previously achieved thermally through heating of the surrounding aqueous medium. In the present invention, the present inventors demonstrate the novel use of a near-infrared (NIR) optically absorbing naphthalocyanine dye (zinc 2,11,20,29-tetra-tert-butyl-2,3-naphthalocynanine) into the solid hydrocarbon (eicosane, n-C20H42) core of liquid fluorocarbon (C₅F₁₂) drops, which can be suspended in an aqueous medium. In one embodiment, photoacoustic endoskeletal droplets, also referred to as droplets or endoskeletal droplets, with an approximate uniform diameter of 11.7+0.7 μm were formed using a flow-focusing microfluidic device. The solid hydrocarbon formed a crumpled spherical structure within the liquid fluorocarbon droplet. The photo-activation behavior of these dye-containing endoskeletal droplets was investigated using NIR laser irradiation. When exposed to a pulsed laser of 720 nm wavelength, the dye-containing droplets vaporized at an average laser fluence of 65 mJ/cm2, whereas blank droplets without dye did not vaporize at any fluence up to 100 mJ/cm2. Further, dye-loaded droplets with a smaller, polydisperse size distribution were prepared using a simple shaking method and studied in a flow phantom for their photoacoustic signal and ultrasound contrast imaging. These results demonstrate that dye-containing endoskeletal droplets can be made to vaporize by externally applied optical energy. Such droplets may be useful for a variety of photoacoustic applications for sensing, imaging and therapy.

In one embodiment, the invention includes a novel photoacoustic endoskeletal droplet composition comprising a solid phase hydrocarbon (HC) core having a near infra-red (NIR) modulating composition surrounded by a liquid phase fluorocarbon (FC). In a preferred embodiment, a near infra-red (NIR) modulating composition of the invention may include a NIR absorbing composition, and more preferably naphthalocyanine dye.

The solid phase HC core of a photoacoustic endoskeletal droplet of the invention can comprise an alkane, and preferably a straight chain alkane, The term “alkane” means substantially saturated compounds containing hydrogen and carbon only, e.g., those containing ≤1% (molar basis) of unsaturated carbon atoms. The term alkane encompasses linear, iso, and cyclo alkanes. The term “alkene” means a straight chain or branched, noncyclic or cyclic, unsaturated aliphatic hydrocarbon having at least one carbon-carbon double bond.

In a preferred embodiment, the straight chain alkane of the invention can preferably include octadecane, eicosane, docosane, tetracosane, or a mixture of the same. Additionally, the liquid phase FC a photoacoustic endoskeletal droplet of the invention can include, but not be limited to: a perfluoroalkane, a perfluoroalkene, a perfluorocycloalkanes, a perfluoro amine, or a combination of the same. In another embodiment, the liquid phase FC a photoacoustic endoskeletal droplet of the invention can include, but not be limited to: perfluoropentane, perfluorohexane, perfluorononane, perfluorohexyl bromide, perfluorooctyl bromide, and perfluorodecyl bromide; perfluoroalkyl ethers; bisperfluorobutylethylene; perfluorodecalin, perfluorocyclohexanes, perfluoroadamantane, perfluorobicyclodecane, perfluoromethyl decahydroquinoline; and C₁-C₈ substituted compounds thereof, isomers thereof, and combinations thereof. The term “perfluo” means a group or compound in which all hydrogen atoms in a CH bond have been replaced by a CF bond.

In one embodiment, the invention includes a novel photoacoustic endoskeletal droplet composition including a solid core including a solid phase hydrocarbon (HC) comprising eicosane, docosane, or a combination of the same, and zinc 2,11,20,29-tetra-tert-butyl-2,3-naphthalocynanine (naphthalocyanine dye), and a liquid phase fluorocarbon (FC), preferably comprising perfluoropentane, surrounding the core.

Additional embodiments of the invention may include pharmaceutical compositions comprising a therapeutically effective amount of photoacoustic endoskeletal droplets of the invention, and a pharmaceutically acceptable carrier/excipient. The term “therapeutically effective amount” means an amount effective to produce a detectable physiological effect, which may be therapeutic or diagnostic in nature. A pharmaceutically acceptable excipients may be included to increase the chemical and/or colloidal stability of the droplets. Preferred excipients are known in the art but may include, but not be limited to chelating agents such as EDTA or its salts such as disodium EDTA, desferoxamine, lipoic acid, glutathione, or antioxidants such as ascorbic acid, ascorbic acid palmitate, alpha-tocopherol, ubiquinol, β-carotene, retinol, lipoic acid, glutathione, and combinations of hydrophilic and/or lipophilic antioxidants as well as chelators. More preferred are excipients selected from the group consisting of EDTA, desferoxamine, glutathione, ubiquinol, hydrophilic antioxidants, ascorbic acid palmitate, and alpha-tocopherol. Most preferred are excipients selected from the group consisting of EDTA, desferoxamine, glutathione, ubiquinol, and hydrophilic antioxidants.

The invention further includes methods of producing a photoacoustic endoskeletal droplet, and in particular populations of poly-and monodisperse photoacoustic endoskeletal droplets. The term “monodisperse” means that a droplet size and shape are highly uniform. As an example, the coefficient of variation (CV), which is defined by the ratio of the standard deviation to the average particle size, is typically 15% or less, preferably 10% or less, more preferably 5% or less. The term “polydisperse” means that a droplet size and shape having a relatively wide particle size and shape distribution.

In one preferred embodiment, the invention includes a method producing a population of polydisperse photoacoustic endoskeletal droplets. In this example, a quantity of solid phase hydrocarbon (HC) and a quantity of a near infra-red (NIR) modulating composition, such as preferably naphthalocyanine dye can be heated, to which at least one fluorosurfactant, such as preferably krytox can be added, as well as a quantity of deionized water (DI). A quantity of at least one liquid phase fluorocarbon (FC) can be introduced to the liquefied HC and NIR modulating composition forming a liquid and solid state solution, which can then be heated and further extruded or emulsified, such as by sonication forming a plurality of polydisperse photoacoustic endoskeletal droplets.

In one preferred embodiment, the invention includes a method producing a population of monodisperse photoacoustic endoskeletal droplets. In this example, a quantity a near infra-red (NIR) modulating composition is solubilized in a quantity of a liquified hydrocarbon (HC) forming a HC solution. This HC solution can be injected into a first inlet channel (1) of a microfluidic device (7) of the invention. A quantity of of liquid fluorocarbon (FC) into a second inlet (2) channel of a microfluidic device (7). Additionally, a quantity of deionized water can be injected into a third inlet channel (3) of a microfluidic device (7), all of said injections preferably being simultaneous and in a rate-controlled manner. Within the microfluidic device (7) of the invention, the HC solution, liquid fluorocarbon and deionized water are directed by channels (6) to a flow focusing junction (5) where the components are mixed forming monodisperse photoacoustic endoskeletal droplets having a core comprising a solid-phase HC and a NIR modulating composition surrounded by liquid phase FC. The monodisperse photoacoustic endoskeletal droplets can be extracted from the flow focusing junction through an outlet channel (4), and may preferably include an FC to HC ratio of 2:1.

As noted above, in one embodiment, the present inventors utilized liquid perfluorocarbons, such as perfluoropentane (C₅F₁₂) for the CF phase of the endoskeletal droplets of the invention. Perfluorocarbons are biologically inert materials with relatively high vapor pressure. The presence of one of the strongest intramolecular covalent bonds (C—F) makes it inert to biological and atmospheric processes, volatile owing to weak intermolecular forces, and especially hydrophobic. FCs have thus been used for blood expansion, acoustic droplet vaporization and detection of high-energy particles.

Non-limiting examples of suitable perfluorocarbons for use in invention may include perfluoroalkanes such as perfluoropentane, perfluorohexane, perfluorononane, perfluorohexyl bromide, perfluorooctyl bromide, and perfluorodecyl bromide; perfluoroalkyl ethers; perfluoroalkenes such as bisperfluorobutylethylene; perfluorocycloalkanes such as perfluorodecalin, perfluorocyclohexanes, perfluoroadamantane, perfluorobicyclodecane, and perfluoromethyl decahydroquinoline; perfluoro amines such as perfluoroalkyl amines; and C₁-C₈ substituted compounds thereof, isomers thereof, and combinations thereof.

The term “photoacoustic endoskeletal droplets,” “endoskeletal droplets,” and/or “droplet” as used interchangeably herein refers to a droplet having a solid hydrocarbon phase inner core surrounded by a volatile fluorocarbon as the outer liquid. In one preferred embodiment, a photoacoustic endoskeletal droplet of the invention may further include a secondary constituent of the solid core that effects the vaporization characteristics of the droplet, such as a naphthalocyanine dye as described herein. Photoacoustic endoskeletal droplets can further be isolated, and/or part of an approximately uniform droplet population, namely a monodisperse endoskeletal droplets, or and/or part of a non-uniform droplet population, namely, polydisperse endoskeletal droplets. The average diameter of a droplet, such as a photoacoustic endoskeletal droplets are contemplated to be between from about 0.1 μm to about 20 μm, and preferably approximately . 5 to 12 μm. The average droplet diameter and droplet size distribution can be determined using different methods of generating the droplet, such as through a microfluidic device, and/or a sonication or other physical perturbance.

Although some embodiments of the invention an endoskeletal droplet can consist essentially of a solid hydrocarbon phase inner core surrounded by a volatile fluorocarbon as the outer liquid, as well as an NIR optically absorbing compound, such as a naphthalocyanine dye, other additives can be optionally included. Suitable additives can include, but are not limited to, hydrogels, anti-oxidants, sequestering agents, chelating agents, steroids, anti-coagulants, drugs, carriers, solvents, preservatives, surfactants, wetting agents, and combinations thereof. A perfluorocarbon droplet can also include excipients such as solubility-altering agents (e.g. ethanol, propylene glycol, and sucrose) and polymers (e.g. polycaprylactones and PLGA's), as well as pharmaceutically active compounds.

The term “near infra-red (NIR) modulating composition” means a composition that can be associated within the solid phase hydrocarbon (HC) core of a photoacoustic endoskeletal droplet of the invention and that changes the vaporization profile of the droplet in response to near infra-red (NIR). In a preferred embodiment, a infra-red (NIR) modulating composition comprises zinc 2,11,20,29-tetra-tert-butyl-2,3-naphthalocynanine (naphthalocyanine dye).

### Embodiment 2

### Vaporizable Exoskeletal Droplets

In one embodiment, the invention includes a novel photoacoustic exoskeletal droplet composition comprising a liquid phase fluorocarbon (FC) core surrounded by a solid phase hydrocarbon (HC) shell, which in one preferred embodiment can further include a near infra-red (NIR) modulating composition surrounded by a. In a preferred embodiment, The solid phase HC shell of a photoacoustic exoskeletal droplet of the invention can comprise a straight chain alkane, such as preferably octadecane, eicosane, docosane, tetracosane, or a mixture of the same. Additionally, the liquid phase FC core of a photoacoustic exoskeletal droplet of the invention can include, but not be limited to, a perfluoroalkane, a perfluoroalkene, a perfluorocycloalkanes, a perfluoro amine, or a combination of the same. In another embodiment, the liquid phase FC core a photoacoustic exoskeletal droplet of the invention can include, but not be limited to: perfluoropentane, perfluorohexane, perfluorononane, perfluorohexyl bromide, perfluorooctyl bromide, and perfluorodecyl bromide; perfluoroalkyl ethers; bisperfluorobutylethylene; perfluorodecalin, perfluorocyclohexanes, perfluoroadamantane, perfluorobicyclodecane, perfluoromethyl decahydroquinoline; and C₁-C₈ substituted compounds thereof, isomers thereof, and combinations thereof.

In one embodiment, the invention includes a novel photoacoustic exoskeletal droplet composition including a liquid phase fluorocarbon (FC) core preferably comprising perfluoropentane, surrounded by a solid phase hydrocarbon (HC) shell comprising eicosane, docosane, or a combination of the same>in one embodiment, the solid phase hydrocarbon (HC) shell can further include zinc 2,11,20,29-tetra-tert-butyl-2,3-naphthalocynanine (naphthalocyanine dye).

Additional embodiments of the invention may include pharmaceutical compositions comprising a therapeutically effective amount of photoacoustic exoskeletal droplets of the invention, and a pharmaceutically acceptable carrier/excipient. A pharmaceutically acceptable excipients may be included to increase the chemical and/or colloidal stability of the droplets. Preferred excipients are known in the art but may include, but not be limited to chelating agents such as EDTA or its salts such as disodium EDTA, desferoxamine, lipoic acid, glutathione, or antioxidants such as ascorbic acid, ascorbic acid palmitate, alpha-tocopherol, ubiquinol, β-carotene, retinol, lipoic acid, glutathione, and combinations of hydrophilic and/or lipophilic antioxidants as well as chelators. More preferred are excipients selected from the group consisting of EDTA, desferoxamine, glutathione, ubiquinol, hydrophilic antioxidants, ascorbic acid palmitate, and alpha-tocopherol. Most preferred are excipients selected from the group consisting of EDTA, desferoxamine, glutathione, ubiquinol, and hydrophilic antioxidants.

The invention further includes methods of producing a photoacoustic exoskeletal droplet, which in a preferred embodiment can include populations of poly-and monodisperse photoacoustic exoskeletal droplets as generally defined herein

In one preferred embodiment, the invention includes a quantity of photoacoustic exoskeletal droplet. In this example, a quantity of solid phase hydrocarbon (HC) is combined with a quantity of liquid phase fluorocarbon (FC) under conditions that form a liquid and solid state solution. In a preferred embodiment, such conditions can include heating and mixing vibrating/shaking the solution. Next, a quantity of a lipid surfactant and a quantity of water, and preferably deionized water, can be added to the to the solution under conditions that form a photoacoustic exoskeletal droplet a liquid phase fluorocarbon (FC) core surrounded by a solid phase hydrocarbon (HC) shell. In a preferred embodiment, the lipid surfactant of the invention can include a phospholipid, and more preferably 1,2-dibehenoyl-sn-glycero-3-phosphocholine (DBPC)

In one preferred embodiment, the invention includes a method producing a population of photoacoustic exoskeletal droplets. In this example, a quantity of liquefied hydrocarbon (HC) can be injected into a first inlet channel (1) of a microfluidic device (7) of the invention, while a quantity of liquid fluorocarbon (FC) into a second inlet (2) channel of a microfluidic device (7). A quantity of deionized water and lipid surfactant can further be injected into a third inlet channel (3) of a microfluidic device (7), all of said injections preferably being simultaneous and in a rate-controlled manner. Within the microfluidic device (7) of the invention, the liquefied hydrocarbon (HC), liquid fluorocarbon (FC), lipid surfactant, and deionized water are directed by channels (6) to a flow focusing junction (5) where the components are mixed forming photoacoustic exoskeletal droplets having a core comprising a liquid-phase FC surrounded by a sold-phase HC shell. The photoacoustic exoskeletal droplets can preferably include a monodisperse population of droplets, and can further be extracted from the flow focusing junction through an outlet channel (4), and may preferably include an FC to HC ratio of 2:1. In another embodiment, a quantity of a near infra-red (NIR) modulating composition is solubilized in a quantity of a liquified hydrocarbon (HC) forming a HC solution prior to injection into the first inlet channel (1) of a microfluidic device (7) of the invention.

As noted above, in one embodiment, the present inventors utilized liquid perfluorocarbons, such as perfluoropentane (C₅F₁₂) for the CF phase of the exoskeletal droplets of the invention. Perfluorocarbons are biologically inert materials with relatively high vapor pressure. The presence of one of the strongest intramolecular covalent bonds (C—F) makes it inert to biological and atmospheric processes, volatile owing to weak intermolecular forces, and especially hydrophobic. FCs have thus been used for blood expansion, acoustic droplet vaporization and detection of high-energy particles.

Non-limiting examples of suitable perfluorocarbons for use in invention may include perfluoroalkanes such as perfluoropentane, perfluorohexane, perfluorononane, perfluorohexyl bromide, perfluorooctyl bromide, and perfluorodecyl bromide; perfluoroalkyl ethers; perfluoroalkenes such as bisperfluorobutylethylene; perfluorocycloalkanes such as perfluorodecalin, perfluorocyclohexanes, perfluoroadamantane, perfluorobicyclodecane, and perfluoromethyl decahydroquinoline; perfluoro amines such as perfluoroalkyl amines; and C₁-C₈ substituted compounds thereof, isomers thereof, and combinations thereof.

The term “photoacoustic exoskeletal droplets,” “exoskeletal droplets,” and/or “droplet” as used interchangeably herein refers to a droplet having a volatile fluorocarbon (FC) core surrounded by a solid hydrocarbon (HC) phase shell. In one preferred embodiment, a photoacoustic endoskeletal droplet of the invention may further include a secondary constituent of the solid shell that effects the vaporization characteristics of the droplet, such as a naphthalocyanine dye as described herein. Photoacoustic endoskeletal droplets can further be isolated, and/or part of an approximately uniform droplet population, namely a monodisperse endoskeletal droplets, or and/or part of a non-uniform droplet population, namely, polydisperse endoskeletal droplets. The average diameter of a droplet, such as a photoacoustic endoskeletal droplets are contemplated to be between from about 0.1 μm to about 20 μm, and preferably approximately. 5 to 12 μm. The average droplet diameter and droplet size distribution can be determined using different methods of generating the droplet, such as through a microfluidic device, and/or a sonication or other physical perturbance.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

As used herein and in the appended claims, the singular forms “a”, “and”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Materials and Methods

Design and fabrication of the microfluidic device: A microfluidic device with three inlet and one outlet junction was fabricated to prepare droplets of uniform size (FIG. 2). Fabrication of the microfluidic device was conducted using two major processes: (1) photolithography and (2) soft lithography. For photolithography, the designs for photomasks were done using Cle Win4 Layout Editor version 4.0.3 (WeiWeb, Hengelo, The Netherlands). The photomasks were printed at CAD/Art Services (Bandon, OR). A layer of positive SU-8 2010 photoresist (Kayaku Advanced Materials, Inc. Westborough, MA) was spin-coated on a silicon wafer (El-Cat Inc., Ridgefield Park, NJ, USA). The pattern on the photomask was transferred to the silicon wafer with a mask exposer (MJB4, KARL SUSS, Germany) and developed in the SU-8 photoresist developer (Kayaku Advanced Materials, Inc, Westborough, MA). The substrate was then dry etched using SF₆ plasma (PlasmaSTAR, AXIC Inc., Santa Clara, CA) to form the final silicon mold. The surface of the silicon mold was silanized using vapor deposition of trichloro(1H,1H,2H,2H-perfluorooctyl) silane. For soft lithography, the PDMS pre-polymers (Sylgard 184 Silicone Elastomer, Dow, Midland, MI) were mixed in ratio of base: curing agent of 10:1. The mixture was then degassed in a vacuum desiccator for 30 min and poured onto the silanized silicon mold and cured at 65° C. overnight to cast the flow channels onto the PDMS. After curing, the PDMS layer was peeled from the silicon mold surface, individual microfluidic PDMS devices were cut out, and inlet and outlet holes were punched. Glass slides were washed with DI water as well as isopropyl alcohol, and any residue was removed using masking tape. The glass slides and individual microfluidic PDMS devices were treated with air plasma using a plasma cleaner (PDC-001,Harrick Plasma, Ithaca, NY, USA) at a pressure of 0.2 Torr at medium intensity for 1 min. Both components were removed from the plasma cleaner, and the surfaces were brought in contact with each other for bonding. The device outlet flow channels were subjected to a hydrophilic surface treatment using a procedure previously described by Trantidou et al. 21 An 1% PVA solution was injected into the outlet channel and left for 15 min after the plasma treatment and bonding. PVA solution was then removed, and the devices were dried on a hotplate at 115° C. for 15 min. The devices were then baked in an oven at 65° C. overnight before using.

Preparation of monodisperse endoskeletal droplets: Two types of monodisperse endoskeletal droplets were synthesized: (1) dye-loaded endoskeletal droplets and (2) endoskeletal droplets without dye (henceforth referred to as blank). For the dye-loaded endoskeletal droplets, 400 mg of eicosane was weighed in a glass vial and heated on a hotplate at 100° C. until all the HC melted. 4 mg (1% by wt. of eicosane) of the dye, ZnNc, was weighed in a separate glass vial, and 500μL of methanol was added to the vial. The vial was kept in a bath sonicator for 2 min so that the dye completely dissolved in methanol. The dye-methanol solution was added dropwise to the melted eicosane maintained at ˜65° C., the boiling point of methanol, and the heated mixture was stirred until all the methanol evaporated and the dye was solubilized in the eicosane. The dye-eicosane mixture was then collected in a 500 μL glass syringe (Gastight 1750, Hamilton, Reno, NV). For the blank droplets, a similar procedure was followed to prepare the HC component, i.e., 400 mg of HC (eicosane) was heated in a water bath until it melted and collected in another 500 μL glass syringe (Gastight 1750, Hamilton, Reno, NV), except the dye was not added to the HC. The endoskeletal droplets (blank and dye-loaded) were synthesized by a flow-focusing technique using the microfluidic PDMS device (FIG. 2). The syringe having hydrocarbon was connected to the HC inlet channel labeled as “1” in FIG. 2 of the microfluidic PDMS device using flexible Tygon tubing (OD 0.07 inches and ID 0.04 inches) and stainless-steel tubing (18G, 204 SS, Component Supply, Sparta, TN). After connecting, the syringe was fixed onto a syringe pump (GenieTouch, Kent Scientific). The hydrocarbon syringe was continuously heated using a tape syringe heater (Syringe Heater, New Era Pump Systems, Farmingdale. NY) set at 45° C. to keep the dye-eicosane mixture (for dye-loaded droplets) or eicosane (for blank droplets) in a melted state. The connecting flexible tube was heated externally using a heat lamp (BR40 incandescent heat lamp, 125 W, GE). The part of the microfluidic PDMS device containing the hydrocarbon inlet was placed on top of a flexible resistance heater (Kapton KHLV-102/10-P, Omega Engineering, Norwalk, CT, USA) attached to a power supply (Agilent E3640A, Agilent Technologies, Santa Clara, CA, USA) set at 6.00 V and delivering 0.157 A to continuously heat the hydrocarbon inlet. A mixture of the fluorocarbon (FC) phase (perfluoropentane, C₅F₁₂) and Krytox (3% v/v of C₅F₁₂) was filled in a different 500 μL glass syringe (Gastight 1750, Hamilton, Reno, NV) and connected to the FC inlet channel labeled as “2” in FIG. 2 of the microfluidic PDMS device using Tygon and stainless-steel tubing. Ultrapure DI water was placed in another 10 mL glass syringe (Gastight 1010, Hamilton, Reno, NV) and connected to the water inlet channel labeled “3” of the microfluidic PDMS device using Tygon and stainless-steel tubing. Both the water and FC syringes were fixed onto another syringe pump (Fusion 4000, Chemyx, Stafford, TX). DI water, FC phase and HC phase were dispensed at flowrates of 20 μL/min, 1 μL/min and 0.5 L/min respectively to synthesize the droplets that were collected from the outlet channel in a glass vial. Because of the flowrates of FC and HC phases, the final droplets had a FC to HC ratio of 2:1. The droplets were collected through outlet channel (labeled “4”) of the microfluidic device in a collection vial.

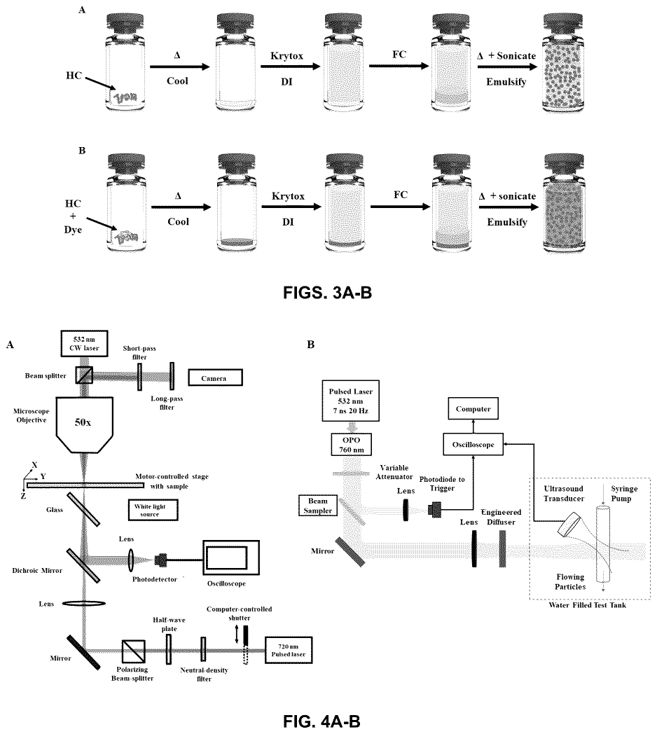

Synthesis of endoskeletal droplets by shaking: Smaller, polydisperse endoskeletal droplets were also prepared by a simple shaking method, following a similar method as used by Shakya et. al. (2020), as shown in FIG. 3.⁹ Briefly, 60 mg hydrocarbon (docosane, C₂₂H₄₆) and 1.2 mg zinc-naphthalocyanine dye (2% by weight of HC) were weighed in a 4 mL serum glass vial. The raw HC and dye were in solid powdered form. The vial containing HC and dye was heated to 47° C. (melting temperature of HC ˜38° C.) in a water bath to melt the HC phase, followed by quenching in an ice bath to form a solid film. The fluoro-surfactant Krytox was added to the vial at 0.75% (v/v), followed by addition of the chilled DI water (4 mL). The vial was cooled by placing it in an ice water bath. The liquid fluorocarbon C₅F₁₂ (76 μL) was pipetted into the cooled vial. The vial was immediately sealed using an aluminum cap and crimped (Wheaton, Millville, NJ, USA). The sealed vial was then heated to 47° C., followed by bath sonication for 1 min. The sonication helped to homogenously mix the melted hydrocarbon and the liquid fluorocarbon present in the aqueous suspension. The sonicated HC/FC/aqueous suspension was then subjected to shaking at 4530 rpm for 45 sec (VialMix, Lantheus, N. Billerica, MA, USA). The resulting emulsified suspension of endoskeletal droplets was then quenched immediately in an ice bath. Control (blank) droplets were prepared in the similar manner, except there was no dye added to the formulation. Prepared dye-loaded and blank droplets were stored at 4° C. for further studies.

Microscopy for droplet visualization: The dye-loaded or blank droplets were diluted in DI water and placed on a glass slide. The droplets were then visualized under the microscope (Olympus BX52, Olympus America Inc., NY, USA) using a 50× objective and imaged using QIClick monochrome camera (Teledyne Photometrics, AZ, USA).

Sizing and counting of the droplets: The size distribution and concentration of the endoskeletal droplets were measured using an Accusizer 780A (PSS Nicomp, Port Richey, FL, USA). The sizing data were imported to OriginPro (OriginLab, Northampton, MA, USA) for analysis and plotting.

Spectrophotometry: To measure the extinction spectra of the monodisperse droplets and confirm the dye encapsulation, a UV-Vis (200 to 999 nm) plate reader (BioTek EPOCH) was used. Four different samples were evaluated: 1) dye-loaded droplets at a concentration of 10⁷ droplets/mL, 2) blank droplets at a concentration of 10⁷ droplets/mL, 3) DI water and 4) dye dissolved in DI water. The dye concentration of this sample was equivalent to the dye-loaded droplets sample. This was calculated using the average droplet diameter of 11.7 μm, FC: HC volume ratio of 2:1, and 2 wt % dye to HC.

Single droplet vaporization: FIG. 4A shows a schematic of the experimental setup to observe the vaporization behavior of individual monodisperse droplets. The experimental setup consisted of a continuous wave (CW) laser operating at 532 nm for detection and a pulsed laser operating at 720 nm for excitation. A long working distance microscope objective (50×, NA=0.42) coupled with a charged coupled device (CCD) camera was used in bright field to image the focal plane of the sample. The 1/e diameter of the detection beam was measured with the knife-edge technique to be 238.75 μm. A combination of motorized half-wave plate and a polarizing beam splitter was used to precisely step through different pulse energies. To further reduce the laser energy, a neutral density (ND) filter (OD=0.75011) was used to obtain a fluence range from 10-160 mJ/cm². A computer-controlled shutter was used to open and close the pulsed excitation beam during the fluence scan, and to stop the beam from illuminating the sample as the fluence value was changed. A long-pass dichroic mirror with a 550 nm cutoff was also incorporated in the setup. This mirror transmitted the pulsed excitation beam towards the droplet sample while reflecting the CW detection beam towards the photodetector. The CW laser power incident on a broadband photodetector was set to 2.5 mW. The direct current (DC) voltage output from the photodetector was digitized with an oscilloscope with a sampling frequency of 1 MHz. The average voltage was then computed from the DC offset value for 100 laser shots for each fluence value. The droplet sample was housed between a microscope glass slide and a coverslip and surrounded by the walls of an incubation chamber sticker well. This slide was mounted on a 2D motor-controlled stage to control the position of an individual droplet. A long-pass and short-pass filter with cutoffs at 540 nm and 700 nm, respectively, were used to filter the CW and pulsed laser from reaching the camera and saturating the field of view.

Photoacoustic detection of endoskeletal droplets: FIG. 4B shows a schematic of the photoacoustic setup used to study polydisperse droplets. A pulsed laser source of 532 nm (20 Hz) was converted to 760 nm using an optical parametric oscillator (OPO). The incident laser energy was varied using an attenuator. Here, the present inventors only examined a laser fluence up to 25 mJ/cm². A beam splitter was used to send a reference beam to the photodiode trigger, which was connected to an oscilloscope. The photodiode triggered the oscilloscope to start collecting the signal as soon as the laser was fired. A mirror was used to reflect the laser to the Tygon tube (OD 0.07 and ID 0.04 inches) through which the droplet sample was flowing. A diffuser was used to ensure the uniform distribution of laser energy on the droplet suspension flowing through tube. The droplet sample was flowed through a 100-nm tube at 0.1 mL/min using a syringe pump. A single-element ultrasound transducer (central frequency 20 MHz, focal length 12.5 mm) set on a 3-axis motorized stage was used to receive the photoacoustic signal from the tube. The stage was centered to get a maximum signal from the tube, and the delay time was calculated based on the distance between the transducer and tube and the speed of sound (˜1500 m/s). The delay time correction was further compensated via the oscilloscope. Data points were collected by sending thirteen different laser fluences. At each fluence value one hundred laser shots were fired. The ultrasound signal was post-processed using a custom MATLAB code, which averaged the signals and calculated the resulting peak-to-peak voltage.

Ultrasound imaging of laser-activated endoskeletal droplets: The vaporization of polydisperse droplets was also imaged using ultrasound. The experimental setup was similar to the previous section, except a clinical ultrasound scanner (Acuson Sequoia 512, Siemens, USA) and Acuson 15L8 transducer was used to collect ultrasound images at 14 MHz and 0.2 mechanical index (MI). A dialysis tube (dry cylindrical diameter 6.37 mm) was connected through Tygon tubing (OD 0.07 and ID 0.04 inches) to a syringe mounted on a syringe pump (GenieTouch, Kent Scientific), and the droplet suspension was pumped at a flow rate of 1 mL/min. The droplet concentration used was ˜10⁹/mL. The acoustically transparent dialysis tubing was submerged in the water tank. An acoustic absorber attached to the water chamber walls was used to avoid reflections. The photoacoustic setup was used to expose droplet suspension with a laser fluence of 25 mJ/cm².

Materials: The following chemicals were used for the inventive embodiments disclosed herein: eicosane (n-C₂₀H₄₂) and docosane (n-C₂₂H₄₆) were purchased from Sigma-Aldrich (St. Louis, MO, USA); perfluoropentane (n-C₅F₁₂, 99%, FluoroMed, Round Rock, TX, USA); Zinc 2,11,20,29-tetra-tert-butyl-2,3-naphthalocyanine (ZnNc, Sigma-Aldrich, St. Louis, MO, USA); Krytox 157 FSH oil (Miller-Stephenson Chemicals, Danbury, CT, USA); polydimethylsiloxane (PDMS) (Sylgard 184 Silicone Elastomer, Dow, Midland, MI); trichloro(1H,1H,2H,2H-perfluorooctyl)silane, polyvinyl alcohol (PVA) (Sigma Aldrich, St. Louis, MO); ultrapure deionized (DI) water from Millipore Direct-Q (Millipore Sigma, St. Louis, MO, USA); SU-8 2010 photoresist, SU-8 photoresist developer (Kayaku Advanced Materials, Inc, Westborough, MA).

### Example 2: Droplet Synthesis and Characterization

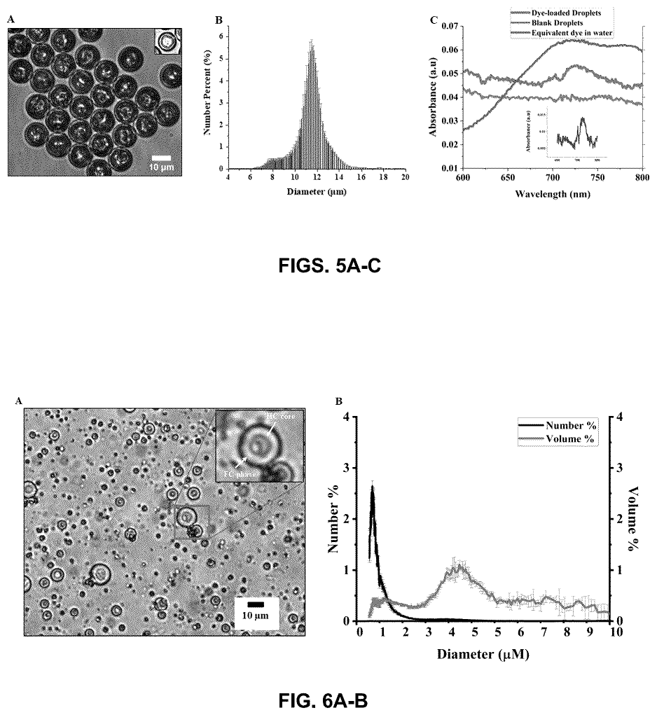

Monodisperse endoskeletal droplets with a solid-hydrocarbon core and liquid-fluorocarbon shell were synthesized using a microfluidic device (FIG. 2). The droplets synthesized using microfluidics were collected from the outlet (labeled “4” in FIG. 2) at a temperature of approximately 38° C., which is slightly higher than the melting point of the eicosane core. Hence, the freshly collected droplets had all three phases in liquid form (not shown). The eicosane solidified as the collected sample cooled. An image of the dye-loaded droplet sample is shown in FIG. 5A. The droplets consisted of a solid core having eicosane (n-C₂₀H₄₂) and ZnNc dye inside the perfluoropentane (n-C₅F₁₂) liquid droplet, stabilized by Krytox and suspended in DI water.

The major advantage of using microfluidics to generate these droplets was the ability to precisely control the size of the droplets and the relative amounts of the HC and FC present in them. The orifice width of the microfluidic device was 10 μm (FIG. 2c). The final droplet size was manipulated within a few micrometers by varying the flowrates of the different liquid components. The uniformity in size distribution of the synthesized droplets is shown in FIG. 5B. The average droplet diameter was 11.7±0.7 μm.

The dye content inside the monodisperse droplets was confirmed using spectrophotometry. The inset in FIG. 5C shows the absorbance spectra of dye-containing endoskeletal droplets normalized against blank endoskeletal droplets. For comparison, only dye dissolved in water was also analyzed for absorbance. As can be observed from FIG. 5C, only the dye sample and the dye-containing endoskeletal droplets had significant absorbance at ˜720 nm wavelength, confirming the presence of dye inside the endoskeletal droplets. Polydisperse endoskeletal droplets were prepared using the shaking method, shown in FIG. 3. The droplets had endoskeletal structures, with an HC core dispersed within the FC droplet (FIG. 6A). As expected, these droplets had more diverse structures and sizes than those made by microfluidics. The mean number-weighted diameter was 0.75±0.026 μm, and the polydispersity was evident by the volume-weighted distribution (FIG. 6B). The blank droplets made by shaking had a comparable size distribution.

### Example 2: Vaporization of Individual Droplets

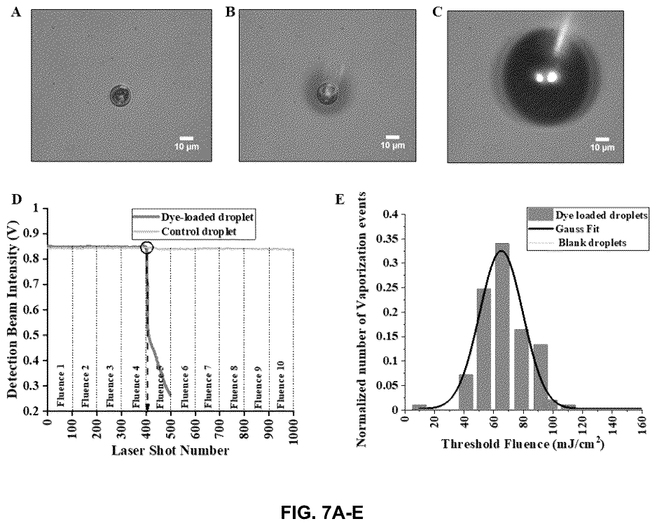

To explore the photoacoustic vaporizability of the endoskeleton droplets, a diluted sample of synthesized droplets containing ˜10⁶ droplets/mL was collected in a cultural cell container on a microscopic slide. An isolated droplet was brought into the field of view as shown in FIG. 7A. The pulsed laser spot was translated at 25 microns in the imaging plane away from the detection beam. This offset distance was necessary because the droplets rotated when they were aligned to the detection beam spot. Motorized stages in the XY plane were used to align the droplet to the focal spot of the pulsed laser. As the energy of the pulsed laser incident on the droplet approached the vaporization threshold, the droplets vaporized into bubbles, as shown in FIG. 7C. The vaporized droplets scattered the CW detection laser, and thus the photodetector intensity dropped, as shown in FIG. 7D. A total of 300 individual dye-loaded endoskeletal droplets were studied in the same manner, leading to all but 11 droplets vaporized to bubbles. The results are presented in a histogram against the 720 nm laser fluence (FIG. 7E). The median fluence for vaporization was determined to be ˜65 mJ/cm². Blank endoskeletal droplets (n=60) without dye encapsulation did not vaporize at any fluence up to 100 mJ/cm².

### Example 3: Photoacoustic Measurements

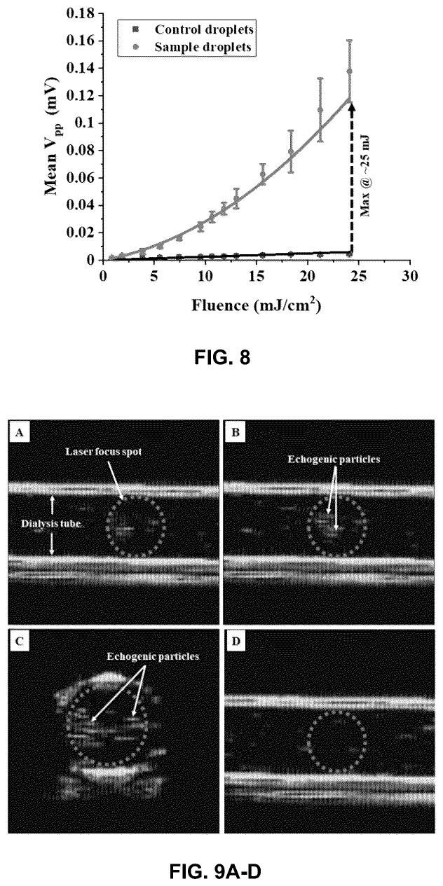

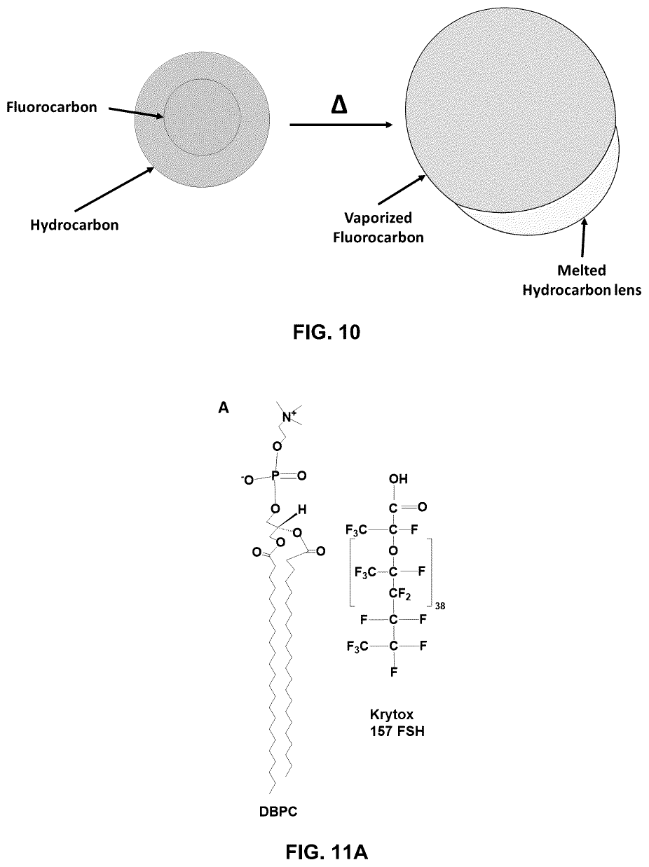

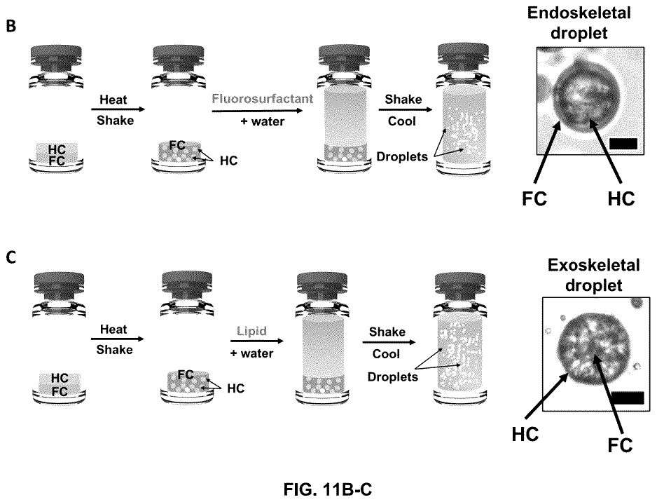

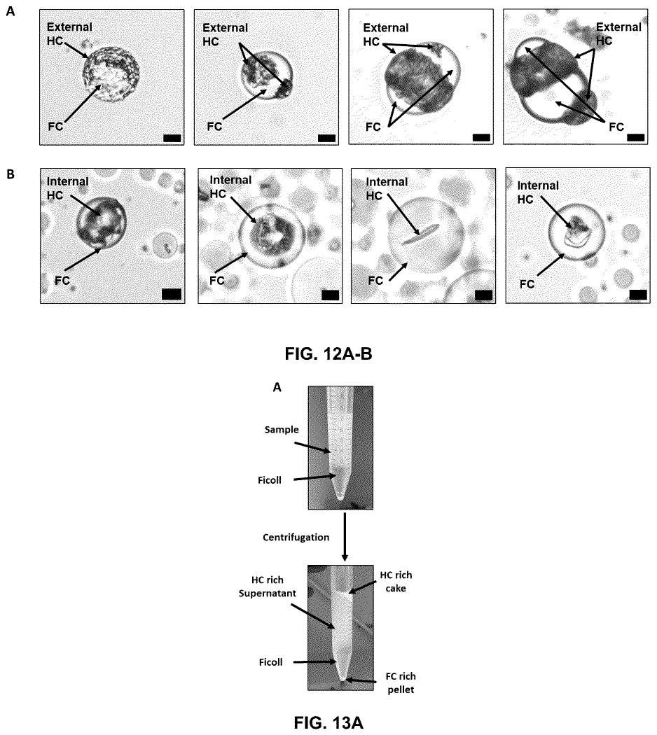

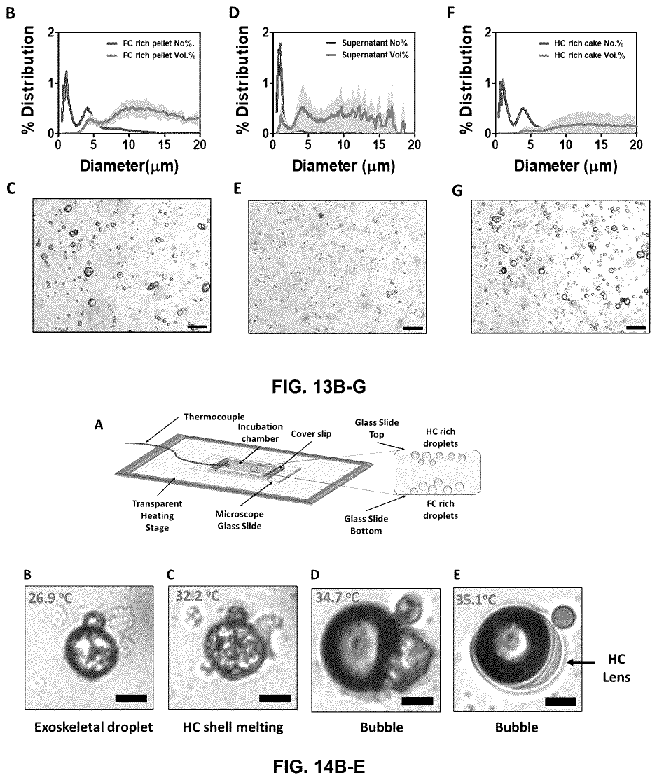

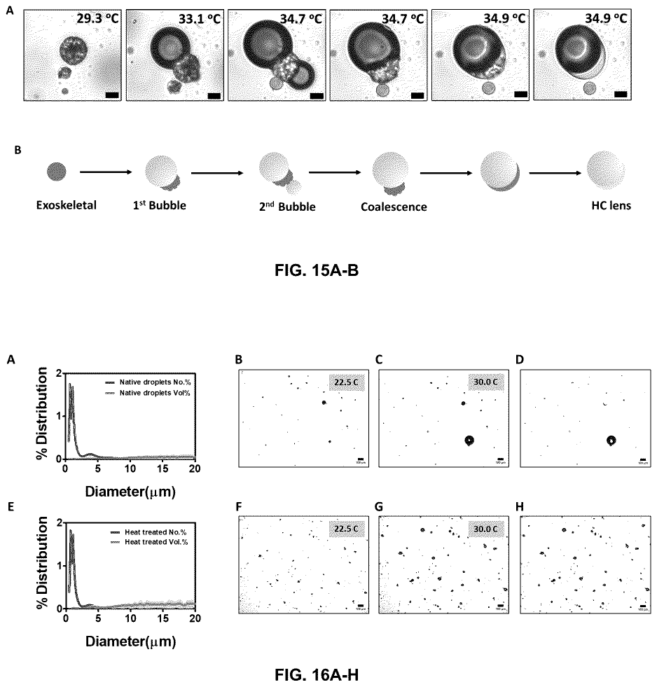

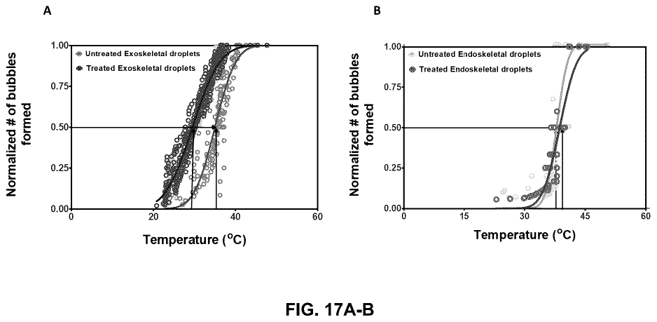

The photoacoustic signal from polydisperse droplets was measured under continuous flow using the setup shown in FIG. 4B. The droplets flowing in the tube were exposed to increasing laser fluence, and their acoustic response was measured by a single-element ultrasound transducer. For both the blank and dye-loaded droplets, increasing acoustic signal was observed with increasing laser fluence (FIG. 8). However, the signal from the dye-loaded droplets was significantly higher and nonlinear.

### Example 4: Ultrasound Imaging of Droplet Vaporization

The present inventors used a clinical ultrasound imaging scanner to examine the echogenicity of the polydisperse droplets upon exposure to the 720-nm laser at 25 mJ/cm². Enhancement of the ultrasound image was observed as bright speckles when the laser was active (FIG. 9A-C). The ultrasound imaging enhancement was not visible for blank droplets (FIG. 9D). The enhancement may be due to droplet vaporization and bubble formation during laser exposure and acoustic insonation. The fluence used here (25 mJ/cm²) for polydisperse droplets was less than the threshold fluence determined above for larger monodisperse droplets (˜65 mJ/cm²). The present inventors speculated that the smaller polydisperse droplets, which are also nonuniform in structure owing to the stochastic nature of the shaking method used to form them (FIG. 6A), may have had a distribution of threshold vaporization fluences.

Prior work on thermal vaporization of endoskeletal droplets showed that processing history and microstructure can have a significant effect on their vaporization behavior.¹³ Additionally, the coincidence of the acoustic rarefactional pressure (0.75 MPa peak negative pressure) of the ultrasound pulse may have facilitated vaporization at this lower fluence.²² Taken together, these effects may have led to a lower threshold fluence for vaporization in this circumstance, thus leading to the observed ultrasound imaging enhancement.

### Example 5: Results and Conclusions

The naphthalocyanine dye-loaded endoskeletal droplets, synthesized by microfluidics or shaking, were shown to have a solid hydrocarbon core surrounded by a fluorocarbon liquid, suspended in an aqueous medium. Monodisperse droplets vaporized using a 720-nm laser with fluences near 65 mJ/cm². The polydisperse droplets showed a strong, nonlinear photoacoustic signal, and they could be observed with ultrasound imaging when stimulated by the laser pulse. These droplets may be useful for photoacoustic imaging and sensing, as well as photothermal therapy.

### REFERENCES

ADDIN ZOTERO_BIBL {“uncited”: [],“omitted”:[],“custom”:[]} CSL_BIBLIOGRAPHY (1) Kripfgans, O. D.; Fowlkes, J. B.; Miller, D. L.; Eldevik, O. P.; Carson, P. L. Acoustic Droplet Vaporization for Therapeutic and Diagnostic Applications. Ultrasound Med Biol 2000, 26 (7), 1177-1189. https://doi.org/10.1016/s0301-5629(00)00262-3.
(2) Sheeran, P. S.; Dayton, P. A. Phase-Change Contrast Agents for Imaging and Therapy. Curr Pharm Des 2012, 18 (15), 2152-2165. https://doi.org/10.2174/138161212800099883.
(3) Borden, M. A.; Shakya, G.; Upadhyay, A.; Song, K.-H. Acoustic Nanodrops for Biomedical Applications. Current Opinion in Colloid & Interface Science 2020, 50, 101383. https://doi.org/10.1016/j.cocis.2020.08.008.
(4) Faraj, K.; Khaled, M.; Faraj, J.; Hachem, F.; Castelain, C. A Review on Phase Change Materials for Thermal Energy Storage in Buildings: Heating and Hybrid Applications. Journal of Energy Storage 2021, 33, 101913. https://doi.org/10.1016/j.est.2020.101913.
(5) Theberge, A. B.; Courtois, F.; Schaerli, Y.; Fischlechner, M.; Abell, C.; Hollfelder, F.; Huck, W. T. S. Microdroplets in Microfluidics: An Evolving Platform for Discoveries in Chemistry and Biology. Angewandte Chemie International Edition 2010, 49 (34), 5846-5868. https://doi.org/10.1002/anie.200906653.
(6) Durham, P. G.; Dayton, P. A. Applications of Sub-Micron Low-Boiling Point Phase Change Contrast Agents for Ultrasound Imaging and Therapy. Current Opinion in Colloid & Interface Science 2021, 56, 101498. https://doi.org/10.1016/j.cocis.2021.101498.
(7) Dove, J. D.; Mountford, P. A.; Murray, T. W.; Borden, M. A. Engineering Optically Triggered Droplets for Photoacoustic Imaging and Therapy. Biomed. Opt. Express, BOE 2014, 5 (12), 4417-4427. https://doi.org/10.1364/BOE.5.004417.
(8) Kuriakose, M.; Borden, M. A. Microbubbles and Nanodrops for Photoacoustic Tomography. Current Opinion in Colloid & Interface Science 2021, 55, 101464. https://doi.org/10.1016/j.cocis.2021.101464.
(9) Mountford, P. A.; Borden, M. A. On the Thermodynamics and Kinetics of Superheated Fluorocarbon Phase-Change Agents. Adv Colloid Interface Sci 2016, 237, 15-27. https://doi.org/10.1016/j.cis.2016.08.007.
(10) Brambila, C. J.; Lux, J.; Mattrey, R. F.; Boyd, D.; Borden, M. A.; de Gracia Lux, C. Bubble Inflation Using Phase-Change Perfluorocarbon Nanodroplets as a Strategy for Enhanced Ultrasound Imaging and Therapy. Langmuir 2020, 36 (11), 2954-2965. https://doi.org/10.1021/acs. langmuir.9b03647.
(11) Shakya, G.; Hoff, S. E.; Wang, S.; Heinz, H.; Ding, X.; Borden, M. A. Vaporizable Endoskeletal Droplets via Tunable Interfacial Melting Transitions. Sci Adv 2020, 6 (14), eaaz7188. https://doi.org/10.1126/sciadv.aaz7188.
(12) Shakya, G.; Yang, T.; Gao, Y.; Fajrial, A. K.; Li, B.; Ruzzene, M.; Borden, M. A.; Ding, X. Acoustically Manipulating Internal Structure of Disk-in-Sphere Endoskeletal Droplets. Nat Commun 2022, 13 (1), 987. https://doi.org/10.1038/s41467-022-28574-4.
(13) Shakya, G.; Fajrial, A. K.; Ding, X.; Borden, M. A. Effect of Thermal History and Hydrocarbon Core Size on Perfluorocarbon Endoskeletal Droplet Vaporization. Langmuir 2022, 38 (8), 2634-2641. https://doi.org/10.1021/acs.langmuir.1c03350.
(14) Wilson, K.; Homan, K.; Emelianov, S. Biomedical Photoacoustics beyond Thermal Expansion Using Triggered Nanodroplet Vaporization for Contrast-Enhanced Imaging. Nat Commun 2012, 3, 618. https://doi.org/10.1038/ncomms1627.
(15) Duffy, M. J.; Planas, O.; Faust, A.; Vogl, T.; Hermann, S.; Schäfers, M.; Nonell, S.; Strassert, C. A. Towards Optimized Naphthalocyanines as Sonochromes for Photoacoustic Imaging in Vivo. Photoacoustics 2018, 9, 49-61. https://doi.org/10.1016/j. pacs.2017.12.001.
(16) Bézière, N.; Ntziachristos, V. Optoacoustic Imaging of Naphthalocyanine: Potential for Contrast Enhancement and Therapy Monitoring. J Nucl Med 2015, 56 (2), 323-328. https://doi.org/10.2967/jnumed.114.147157.
(17) Kumar, A.; George, N.; Nampoori, V. P. N.; Radhakrishnan, P. Spectral Studies of Naphthalocyanine (Ne) and Rare Earth Phthalocyanine (RePc) Molecules in an Inorganic Glassy Borate Matrix. 2000, 41 (4), 5.
(18) Leem, D.; Lee, K.; Li, N.; Park, B. W.; Choi, T.; Ro, T.; Kwon, O. K.; Kwon, Y.; Ng, T. N.; Kim, S. Highly Responsive and Thermally Reliable Near-Infrared Organic Photodiodes Utilizing Naphthalocyanine Molecules Tuned with Axial Ligands. Adv. Optical Mater. 2021, 9 (4), 2001682. https://doi.org/10.1002/adom.202001682.
(19) Yates, N. C.; Moan, J.; Western, A. Water-Soluble Metal Naphthalocyanines—near-IR Photosensitizers: Cellular Uptake, Toxicity and Photosensitizing Properties in Nhik 3025 Human Cancer Cells. Journal of Photochemistry and Photobiology B: Biology 1990, 4 (4), 379-390. https://doi.org/10.1016/1011-1344(90)85017-Q.
(20) El-Khouly, M. E.; Moiseev, A. G.; van der Est, A.; Fukuzumi, S. Photoinduced Electron Transfer in Zinc Naphthalocyanine-Naphthalenediimide Supramolecular Dyads. ChemPhysChem 2012, 13 (5), 1191-1198. https://doi.org/10.1002/cphc.201100948.
(21) Trantidou, T.; Elani, Y.; Parsons, E.; Ces, O. Hydrophilic Surface Modification of PDMS for Droplet Microfluidics Using a Simple, Quick, and Robust Method via PVA Deposition. Microsyst Nanoeng 2017, 3 (1), 16091. https://doi.org/10.1038/micronano.2016.91.
(22) McLaughlan, J. R.; Roy, R. A.; Ju, H.; Murray, T. W. Ultrasonic Enhancement of Photoacoustic Emissions by Nanoparticle-Targeted Cavitation. Opt Lett 2010, 35 (13), 2127-2129. https://doi.org/10.1364/OL.35.002127.

1-49. (canceled)

50. A photoacoustic endoskeletal droplet comprising:
a solid phase hydrocarbon (HC) core having a near infra-red (NIR) modulating composition; and
a liquid phase fluorocarbon (FC) surrounding the core.

51. The droplet of claim 50, wherein said (NIR) modulating composition comprises a NIR absorbing composition.

52. The droplet of claim 51, wherein said NIR absorbing composition comprises zinc 2,11,20,29-tetra-tert-butyl-2,3-naphthalocynanine (naphthalocyanine dye).

53. The droplet of claim 50, wherein said solid phase HC core comprises a straight chain alkane.

54. The droplet of claim 53, wherein said straight chain alkane comprises straight chain alkane selected from: octadecane, eicosane, docosane, tetracosane, and combinations thereof.

55. The droplet of claim 50, wherein said liquid phase FC comprises a liquid phase FC selected from: a perfluoroalkane, a perfluoroalkene, a perfluorocycloalkanes, a perfluoro amine, and combinations thereof.

56. The droplet of claim 50, wherein said liquid phase FC is selected from: perfluoropentane, perfluorohexane, perfluorononane, perfluorohexyl bromide, perfluorooctyl bromide, and perfluorodecyl bromide; perfluoroalkyl ethers; bisperfluorobutylethylene; perfluorodecalin, perfluorocyclohexanes, perfluoroadamantane, perfluorobicyclodecane, perfluoromethyl decahydroquinoline; and C₁-C₈ substituted compounds thereof, isomers thereof, and combinations thereof.

57. The droplet of claim 50, wherein said liquid phase FC is perfluoropentane.

58. The droplet of claim 50, wherein said photoacoustic endoskeletal droplet comprises a plurality of monodisperse endoskeletal droplets.

59. The droplet of claim 50, wherein said photoacoustic endoskeletal droplet comprises a plurality of polydisperse endoskeletal droplets.

60. A photoacoustic endoskeletal droplet comprising:
a solid core including:
a solid phase hydrocarbon (HC);
zinc 2,11,20,29-tetra-tert-butyl-2,3-naphthalocynanine (naphthalocyanine dye); and
a liquid phase fluorocarbon (FC) surrounding the core.

61. The droplet of claim 60, wherein said solid phase HC core comprises a straight chain alkane.

62. The droplet of claim 60, wherein said straight chain alkane comprises straight chain alkane selected from: octadecane, eicosane, docosane, tetracosane, and combinations thereof.

63. The droplet of claim 60, wherein said liquid phase FC comprises a liquid phase FC selected from: a perfluoroalkane, a perfluoroalkene, a perfluorocycloalkanes, a perfluoro amine, and combinations thereof.

64. The droplet of claim 60, wherein said liquid phase FC is selected from: perfluoropentane, perfluorohexane, perfluorononane, perfluorohexyl bromide, perfluorooctyl bromide, and perfluorodecyl bromide; perfluoroalkyl ethers; bisperfluorobutylethylene; perfluorodecalin, perfluorocyclohexanes, perfluoroadamantane, perfluorobicyclodecane, perfluoromethyl decahydroquinoline; and C₁-C₈ substituted compounds thereof, isomers thereof, and combinations thereof.

65. The droplet of claim 60, wherein said liquid phase FC is perfluoropentane.

66. The droplet of claim 60, wherein said photoacoustic endoskeletal droplet comprises a plurality of monodisperse endoskeletal droplets.

67. The droplet of claim 60, wherein said photoacoustic endoskeletal droplet comprises a plurality of polydisperse endoskeletal droplets.

68-72. (canceled)

73. A pharmaceutical composition comprising a photoacoustic endoskeletal droplet of any of claims 50-67, and a pharmaceutically acceptable carrier.

74-99. (canceled)
